(19) United States
(12) Patent Application Publication
(10) Pub. No.: US 2011/0027361 A1
(43) Pub. Date: Feb. 3, 2011

(54) EXTENDED RELEASE DOSAGE FORM OF PALIPERIDONE

(75) Inventors: Ganesan Murugesan, Bhat (IN); Jaya Abraham, Bhat (IN); Vinodkumar Gupta, Bhat (IN); Bhavesh Shah, Bhat (IN)

Correspondence Address: PAULY, DEVRIES SMITH & DEFFNER, L.L.C., Plaza VII-Suite 3000, 45 South Seventh Street, MINNEAPOLIS, MN, 55402-1630, US

(73) Assignee: TORRENT PHARMACEUTICALS LTD., Ahmedabad, Gujarat (IN)

(21) Appl. No.: 12/865,801

(22) PCT Filed: Feb. 3, 2009
(86) PCT No.: PCT/IN2009/000072

§ 371 Date: Sep. 30, 2010

(30) Foreign Application Priority Data
Feb. 4, 2008 (IN) 232/MUM/2008

(51) Int. Cl. A61K 9/28; A61K 9/00; A61K 31/519

(52) U.S. Cl. 424/465; 424/484; 514/259.41; 424/488; 424/485

(57) ABSTRACT

The present invention relates to an extended release solid oral pharmaceutical composition comprising Paliperidone or its pharmaceutically acceptable salts and process for preparing the same.

### FIELD OF THE INVENTION

The present invention relates to an extended release solid oral pharmaceutical composition comprising Paliperidone Or its pharmaceutically acceptable salts and process for preparing the same.

### BACKGROUND OF THE INVENTION

Paliperidone is a psychotropic agent belonging to the chemical class of benzisoxazole derivatives. Its molecular formula is C₂₃H₂₇FN₄O₃ Paliperidone is sparingly soluble in 0.1N HCl and methylene chloride and practically insoluble in water.

Paliperidone is an active metabolite of the older antipsychotic risperidone (paliperidone is 9-hydroxy risperidone, i.e. risperidone with an extra hydroxyl group). An immediate release tablet comprising risperidone is available in market as Risperdal® by Janssen Pharmaceutical Products. A prolong release injectable for risperidone, Risperdal Consta® is also being marketed.

Paliperidone is practically insoluble in water. Further, paliperidone has terminal elimination half-life of approximately 23 hours; which makes its unsuitable candidate for extended delivery. However side effects such as anxiety, somnolence, dizziness, constipation, extrapyramidal symptoms may be related to high blood plasma concentration levels restricting the ability to administer a single daily immediate release dose.

WO 9741837 A1, WO 200189482 A1, U.S. Pat. No. 5,792,477 A, U.S. Pat. No. 5,916,598 A, U.S. Pat. No. 6,110,503, U.S. Pat. No. 6,290,983 A, U.S. Pat. No. 6,403,114 A disclose delivery of risperidone and/or paliperidone through injectable implants for long term, multi-day, delivery.

Many oral osmotic dosage forms of paliperidone are disclosed in WO 2004010981 A1, WO 2006085856 Al, WO 2007 044234 Al, WO 2007 050377 Al. WO2006017537 discloses dosage form which shows ascending rate of release over an extended period of time.

Presently Paliperidone is available as INVEGA® Extended-Release Tablets in 1.5 mg , 3 mg, 6 mg and 9 mg strengths. INVEGA® utilizes OROS® osmotic drug-release technology. Inactive ingredients are carnauba wax, cellulose acetate, hydroxyethyl cellulose, propylene glycol, polyethylene glycol, polyethylene oxides, povidone, sodium chloride, stearic acid, butylated hydroxytoluene, hypromellose, titanium dioxide, and iron oxides. The 3 mg tablets also contain lactose monohydrate and triacetin. INVEGA® (paliperidone) Extended-Release Tablet is indicated for the treatment of schizophrenia.

INVEGA® utilizes osmotic pressure to deliver paliperidone at a controlled rate. The delivery system consists of an osmotically active trilayer core surrounded by a subcoat and semipermeable membrane. The trilayer core is composed of two drug layers containing the drug and excipients, and a push layer containing osmotically active components. There are two precision laser-drilled orifices on the drug-layer of the tablet. In an aqueous environment, such as the gastrointestinal tract, the water-dispersible overcoat erodes rapidly. Water then enters the tablet through the semipermeable membrane that controls the rate at which water ingress the tablet core, which, in turn, determines the rate of drug delivery. The hydrophilic polymers of the core hydrate and swell creating a gel containing paliperidone that is then pushed out through the tablet orifices. The biologically inert components of the tablet remain intact during gastrointestinal transit and are eliminated in the stool as a tablet shell, along with insoluble core components.

There are various disadvantages associated with osmotic drug-release technology; few of them are as below:
1. Osmotic drug-release technology requires highly sophisticated equipments for processes like compression, coating and laser drilling. This ultimately increases the cost of manufacturing.
2. The manufacturing process is critical. INVEGA® is trilayer capsule shape tablet which require atleast two granulation steps and compression into three layers on special compression machine. The triple layer tablet is coated with special membrane i.e semipermeable membrane and then on each tablet two holes are drilled, which has specific size. Overall the entire process is tedious and critical.
3. As the tablet manufactured by osmotic drug-release technology is eliminated in the faeces as a tablet form, along with insoluble core components. This can create panic in patient.
4. There are chances that during laser drilling operation tablet may not get laser drilled, under such condition no drug will be released from the tablet. To avoid such incidences highly sophisticated procedures are performed.
5. Osmotic drug-release technology requires special excipients like osmogen, osmopolymer, polymer for semipermeable membrane, which ultimately increases cost of manufacturing.
Thus there is still unmet need to develop a simple, stable, extended release solid oral pharmaceutical composition of Paliperidone, which does not require such highly precise technique like drilling on the dosage form.
The instant invention has following features:
a) Simple manufacturing process.
b) Acceptable dissolution profile.
c) Acceptable economics of excipients.
d) Acceptable economics of manufacturing process.
e) Stable composition.

### SUMMARY OF THE INVENTION

The first aspect of the present invention is to provide an extended release solid oral pharmaceutical composition comprising Paliperidone or a pharmaceutically acceptable salt thereof.

Another aspect of the present invention is to provide an extended release solid oral pharmaceutical composition of Paliperidone or a pharmaceutically acceptable salt thereof, wherein, the extended release composition is a matrix composition.

Another aspect of the present invention is to provide an extended release solid oral pharmaceutical composition comprising Paliperidone or a pharmaceutically acceptable salt wherein, the extended release formulation is a matrix composition and the matrix agent is selected from insoluble release controlling agents or hydrophilic release controlling agents or fatty release controlling agents or combination thereof.

Another aspect of the present invention is to provide an extended release solid oral pharmaceutical composition comprising Paliperidone or a pharmaceutically acceptable salt thereof wherein, the extended release formulation is a matrix composition coated with coating layer comprising water insoluble release controlling agent.

Another aspect of the present invention is to provide an extended release solid oral pharmaceutical composition comprising Paliperidone or a pharmaceutically acceptable salt thereof wherein, the extended release formulation is a matrix composition coated with coating layer comprising pH dependant water insoluble release controlling agent.

Another aspect of the present invention is to provide an extended release solid oral pharmaceutical composition of Paliperidone or a pharmaceutically acceptable salt thereof wherein, the extended release formulation is a matrix composition coated with coating layer comprising pH independent water insoluble release controlling agent.

Another aspect of the present invention is to provide an extended release solid oral pharmaceutical composition of Paliperidone or a pharmaceutically acceptable salt thereof, wherein the extended release formulation is a matrix composition comprising hydrophilic release controlling agent and this matrix is coated with enteric coating.

Another aspect of the present invention is to provide an extended release solid oral pharmaceutical composition comprising Paliperidone or a pharmaceutically acceptable salt thereof, hydrophilic release controlling agent, wetting agent, which is further coated with enteric coating.

Another aspect of the . present invention is to provide an extended release solid oral pharmaceutical composition comprising Paliperidone or a pharmaceutically acceptable salt thereof, ionic hydrophilic release controlling agent, wetting agent, which is further coated with enteric coating.

Another aspect of the present invention is to provide an extended release solid oral pharmaceutical composition comprising Paliperidone or a pharmaceutically acceptable salt thereof, non ionic hydrophilic release controlling agent, wetting agent, which is further coated with enteric coating.

Another aspect of the present invention is to provide an extended release solid oral pharmaceutical composition comprising Paliperidone or a pharmaceutically acceptable salt thereof, hydrophilic release controlling agent, wetting agent, stabilizing agent, wherein hydrophilic release controlling agent is added in an intragranular or extragranular stage, preferably at intragranular stage.

Another aspect of the present invention is to provide an extended release solid oral pharmaceutical composition comprising Paliperidone or a pharmaceutically acceptable salt thereof, hydrophilic release controlling agent, wetting agent, stabilizing agent, wherein hydrophilic release controlling agent is added in an intragranular or extragranular stage, preferably at intragranular stage, which is further coated with enteric coating.

Another aspect of the present invention is to provide an extended release solid oral pharmaceutical composition of Paliperidone or a pharmaceutically acceptable salt thereof wherein the composition is manufactured by direct compression, wet granulation or compaction process.

Another aspect of the present invention is to provide an extended release solid oral pharmaceutical composition comprising Paliperidone or a pharmaceutically acceptable salt thereof, wherein the particle size of Paliperidone is 90% particles are less than 100 microns; preferably 90% particles are less than 50 microns, more preferably 90% particles are less than 10 microns.

Another aspect of the present invention is to provide the process of preparing an extended release pharmaceutical composition of Paliperidone.

Another aspect of the present invention is to provide an extended release solid oral pharmaceutical composition of Paliperidone or a pharmaceutically acceptable salt thereof, wherein the composition is a combination of different extended release profiles of Paliperidone.

Another aspect of the present invention is to provide a process of preparation of extended release solid oral pharmaceutical composition of Paliperidone or a pharmaceutically acceptable salt thereof, which comprises of following steps:
a) Mixing of paliperidone and atleast one pharmaceutically acceptable excipient. The blend is then granulated, dried and then graded, so as to obtain the granules.
b) Mixing of the granules obtained in step a) with release controlling agent.
OR
a) Mixing of paliperidone, release controlling agent and atleast one pharmaceutically acceptable excipient. The blend is then granulated, dried and then graded, so as to obtain the granules.
b) Lubrication of the mixture obtained in step a) with lubricants.
c) Compression of the lubricated mixture obtained in step c) in to a tablet or filling in to a capsule.
d) Optionally coating the compressed tablets from step d) with water insoluble release controlling agent.

### DETAILED DESCRIPTION OF THE INVENTION

The instant invention discloses an extended release composition of paliperidone by applying simple and conventional formulation technology. Paliperidone as such is very less soluble in aqueous media and has pH dependant solubility. Its solubility is also increased by incorporating wetting agent in the core.

The term “extended release” as used herein refers to a release, which is not immediate release. Further, the term “extended release” as used herein refers to the release of an active ingredient from a pharmaceutical composition or dosage form, in which the active ingredient is released over an extended period of time and/or at particular location and is taken to encompass sustained release, controlled release, modified release, prolonged release, delayed release and the like.

The term “dosage form” or “formulation” or “composition” as used in this specification and the claims refer to physically discrete units, wherein each unit containing a predetermined quantity of active ingredient in association with the required pharmaceutically acceptable excipients. The dosage form used herein selected from tablets, capsule, sachets, pellets, beads, pills, lozenges, or granules.

The use of the terms “a” and “an” and “the” and similar referents in the context of describing the invention are to be construed to cover both the singular and the plural, unless otherwise indicated herein or clearly contradicted by context.

The dosage form of the present invention comprises Paliperidone or its pharmaceutically acceptable salts in a range of about 0.5 mg to about 20 mg; preferably dosage forms may contain 1 mg to about 18 mg, more preferably dosage forms may contain from 1.5 to 12 mg.

The term “Paliperidone” or “drug” or “active ingredient” as used herein includes Paliperidone free base, enantiomers, pharmaceutically acceptable salts, solvates, and polymorphs thereof. The drug may be present in matrix in intragranular stage and/or extragranular stage and/or dissolved or dispersed in binder stage. Optionally the drug may be present in coating stage. The Paliperidone may be present in solid form or dissolved/dispersed in solvents. The paliperidone is present in the dosage form in an amount ranging from 1% to 10% of total weight of dosage form.

According to one embodiment, the .present invention comprises of Paliperidone and atleast one pharmaceutically acceptable excipient.

The pharmaceutically acceptable excipients are selected from the following categories but not limited to diluents, binders, anti-adherents, lubricants, wetting agents, pH modifiers, buffering agents, stabilizers, release controlling agents, film forming polymers, plasticizers, anti tacking agents, pore forming agents and other excipients known to the person skilled in the art.

As used herein, the term “diluents” is intended to mean inert substances used as fillers to create the desired bulk, flow properties, and compression characteristics in the preparation of dosage form. If desired, more than one diluent or fillers can be used. Such compounds include, by way of example and without limitation, dibasic calcium phosphate, lactose anhydrous, lactose monohydrate, pregelatinized starch, mannitol, directly compressible mannitol, Sorbitol, microcrystalline cellulose, powdered cellulose, sorbitol and starch and other materials known to one of ordinary skill in the art. Direct compression grades of diluents can also be selected. The various coprocessed diluents like silicified microcrystalline cellulose, Microcelac® can also be used. The amount of diluent present in the instant invention is in the range from 20% to 90% of total weight of dosage form.

As used herein, the term “binders” is intended to mean inert substance used to form the bridge between the drug particles with other excipients. Binder may be selected from copolyvidone, shellac, starch, hypromellose, hyprolose, povidone, zein, gelatin, polymethacrylates, synthetic resins, Eudragits, cellulose polymers and the like. The amount of binder present in the instant invention is in the range from 0.5% to 5% of total weight of dosage form.

Anti adherents include, by way of example and without limitation, magnesium stearate, talc, calcium stearate, glyceryl behenate, Polyethylene glycols, hydrogenated vegetable oil, mineral oil, stearic acid and other materials known to one of ordinary skill in the art. The amount of anti adherents present in the instant invention is in the range from 0.5% to 2% of total weight of dosage form.

Glidants include cornstarch, talc, calcium silicate, magnesium silicate, colloidal silicon dioxide, silicon hydrogel and other materials known to one of ordinary skill in the art. The amount of glidant present in the instant invention is in the range from 0.25% to 2% of total weight of dosage form.

The term “pore forming agent” as used herein should be able to provide the desired porosity to water insoluble release controlling coat and may be selected from, but not limited to Lactose, Hypromellose, Microcrystalline cellulose, Sorbitol, Magnesium oxide and the like known to person having ordinary skill in the art.

The term “matrix composition” as used herein is related to core composition comprising Paliperidone and atleast one release controlling agent.

Paliperidone is having very less aqueous solubility, which is insufficient to achieve the desired in-vitro dissolution profile and subsequently less bioavailability. So one of the embodiments of the present invention is to increase the solubility of the drug and incorporate such solubility improved drug in extended release dosage form. Various known solubility enhancement techniques can be used to improve the solubility of Paliperidone. Such techniques may be selected from micronization of drug, use of wetting agents, use of solubilizers, complexation with polymers like cyclodextrin, using solid dispersion techniques and the like. The solubility of Paliperidone is enhanced by incorporating wetting agent into the drug core. Wetting agents can be selected from the group of surfactants, solubilizers, complexing agents. The preffered wetting agents are surfactants. Surfactants of the present invention include, but are not limited to anionic and non-ionic surfactants such as sodium lauryl sulfate, poloxamers (copolymers of polyoxyethylene and polyoxypropylene), natural or synthetic lecitins as well as esters of sorbitan and fatty acids, such as Span®, esters of polyoxyethylenesorbitan and fatty acids, such as Polysorbates or Polysorbate® (Commercially available from Spectrum Chemical, Gardena Calif.) polyoxyethylated hydrogenated castor oils, such as Cremophor®, polyoxyethylene stearates, such as Myrj® or any combinations of the surfactants. Preferably the surfactant is a poloxamer 188. The amount of surfactant when present in the instant invention is in the range from 0.1% to 10% of total weight of dosage form.

In another embodiment, the micronized Paliperidone is used to manufacture extended release solid oral pharmaceutical composition of Paliperidone. The particle size of Paliperidone is such that 90% drug particles are less than 100 microns; preferably 90% drug particles are less than 50 microns, more preferably 90% drug particles are less than 10 microns.

Lubricants include, by way of example and without limitation, calcium stearate, magnesium stearate, sodium stearyl fumerate, glyceryl palmitostearate, glyceryl stearate, glyceryl behenate, mineral oil, stearic acid, and zinc stearate and other materials known to one of ordinary skill in the art. The amount of lubricant present in the instant invention is in the range from 0.5% to 3% of total weight of dosage form.

As used herein, the term ‘plasticizer’ should be able to provide the desired plasticity to the coating. Plasticizers, such as propylene glycol, polyethylene glycol, glyceryl monopalmetostearate/Glyceryl monostearate, dibutyl phthalate, triethyl citrate, dibutyl sebacate are included. The plasticizers can be hydrophilic or hydrophobic in nature as known to person having ordinary skill in the art. The amount of plasticizer present in the instant invention is in the range from 0.0% to 50% of total weight of film forming polymer.

The stabilizers as mentioned herein are substances that prevent the decomposition of the active ingredients, which may be due to chemical processes like oxidation, or physical processes like heating. Some of the stabilizers are agents like ascorbic acid, ascorbic palmitate, Vitamin E, butylated hydroxyani sole, butylated hydroxy toluene, hypophosphorous acid, monothioglycerol, propyl gallate, sodium ascorbate, sodium bisulfite, sodium folmaldehyde sulfoxylate, sodium metalbisulfite and other such materials known to those of ordinary skill in the art. The most preferable stabilizer used here is butylated hydroxy toluene. The amount of stabilizer present in the instant invention is in the range from 0.0% to 0.5% of total weight of dosage form.

According to one embodiment, matrix composition of the present invention can be prepared using any or combination of these three types of release controlling agent:
a) Insoluble release controlling agents,
b) Hydrophilic release controlling agents, or
c) Fatty release controlling agents.

Insoluble release controlling agents include without limitation polymethacrylates, polyvinyl chloride, polyvinyl alcohol, ethyl cellulose and polyethylene. Hydrophilic release controlling agents include without limitation methylcellulose, hydroxypropyl methylcellulose (HMPC) and sodium carboxymethylcellulose. Fatty release controlling agents include without limitation various waxes such as carnauba wax and glyceryl tristearate. The amount of release controlling agent in matrix composition is present in the instant invention in the range from 0.5% to 40% of total weight of dosage form. The ratio of active ingredient and release controlling agent in matrix composition present is between 1: 0.5 to 1:20.

The matrix composition comprising hydrophilic release controlling agents is preffered for manufacturing extended release solid oral pharmaceutical composition of Paliperidone.

According to one embodiment, the matrix composition is manufactured by using hydrophilic release controlling agent, which further includes hydroxyethyl cellulose, hydroxypropyl cellulose, sodium alginate, Sodium carboxymethylcellulose, carbomer, xanthan gum, guar gum, locust bean gum. The hydrophillic release controlling agent may be non ionic like HPMC, anionic like Xanthan gum.

The preffered hydrophilic release controlling agent is Hydroxypropyl methylcellulose (HPMC, hypromellose). When HPMC comes in contact with water, HPMC hydrates rapidly and forms a gelatinous barrier layer around the tablet. The rate of drug release from HPMC matrix is dependent on various factors such as type of polymer, drug, polymer/drug ratio, particle size of drug and polymer, and the type and amount of excipients used in the formulation. The preferred hydrophilic release controlling agent that can be used is hydroxypropyl methylcellulose (HPMC) of grades HPMC K15M, HPMC K100M, HPMC K100M CR, and HPMC K4M CR alone or in combination. The hydrophilic release controlling agent is preferably added intragranularly.

According to one embodiment, the matrix composition is coated with water insoluble release controlling coat or hydrophobic release controlling coat.

The water insoluble release controlling coat or hydrophobic release controlling coat comprises of water insoluble or hydrophobic release^{,} controlling agent and optionally hydrophobic or hydrophilic plasticizer, anti-tacking agents, pore forming agents. The coating weight gain is in the range of 2% to 30%.

The water insoluble/hydrophobic release controlling agent is selected from the group consisting of cellulose ether such as ethyl cellulose, polymethacrylates, Kollicoat SR 30D® (an aqueous dispersion composed of 27% polyvinyl acetate (PVAc), 2.5% povidone, and 0.3% sodium lauryl sulfate), polyvinyl alcohol-maleic anhydride copolymers and the like. The commercially available Eudragit® L 100-55, Eudragit® L 30 D-55, Eudragit® L 100, Eudragit® FS 30D, Eudragit® RL, Eudragit® RL 100, Eudragit® RS, Eudragit® NE 30 D, HPMC Phthalate can also be used as water insoluble release controlling agent. The water insoluble release controlling agent may be any enteric coating polymer. The preffered water insoluble/hydrophobic release controlling agent is Eudragit® FS 30D.

The water insoluble release controlling agent may be pH dependent polymer like Eudragit® FS 30D or pH independent polymer like Eudragit® RL 100.

The solvents which can be used in instant invention are inorganic solvent like water, organic solvent like Methanol, Ethanol, Isopropyl alcohol, Acetone, Methylene chloride and the like known to person having ordinary skill in the art. Various cosolvents known to person having ordinary skill in the art can also be used in instant invention.

According to one embodiment, the present invention provides extended release solid oral pharmaceutical composition of Paliperidone or a pharmaceutically acceptable salt thereof, wherein the composition provides two different release profiles, wherein one release profile is delivered by extended release formulation comprising matrix composition coated with coating layer comprising water insoluble release controlling agent and other release profile may be immediate release or extended release in which Paliperidone is present in outer coat.

According to one embodiment, the present invention provides extended release solid oral pharmaceutical composition of Paliperidone or a pharmaceutically acceptable salt thereof, wherein the composition provides atleast two different extended release profiles, wherein each release profile is delivered by extended release formulation comprising Paliperidone pellets coated with coating layer comprising water insoluble release controlling agent. These pellets with pharmaceutically acceptable excipients may be compressed into tablet or filled into capsule. Optionally the tablet may be coated with water insoluble release controlling agent.

According to one embodiment, the present invention provides extended release solid oral pharmaceutical composition of Paliperidone or a pharmaceutically acceptable salt thereof, wherein Paliperidone pellets or tablets having variable release profiles are compressed and optionally coated with water insoluble release controlling coat.

According to one embodiment, the present invention provides extended release solid oral pharmaceutical composition of Paliperidone or a pharmaceutically acceptable salt thereof, wherein pellets comprises of Paliperidone and atleast one release controlling agent, wherein said pellets are manufactured by extrusion-spheronization or prepared by coating over inert core. Said pellets are further coated alternatively by water insoluble release controlling coat and part quantity of Paliperidone. According to this embodiment of present invention, the said composition comprises of atleast one such “sandwiching” of Paliperidone layer between water insoluble release controlling coat. These pellets are compressed into tablet with atleast one pharmaceutically acceptable excipients or filled in to capsule. Tablets are optionally coated with water insoluble release controlling coat.

According to one embodiment, the present invention provides extended release solid oral pharmaceutical composition of Paliperidone or a pharmaceutically acceptable salt thereof, wherein pellets comprising Paliperidone and atleast one release controlling agent, wherein said pellets are manufactured by extrusion-spheronization or prepared by coating over inert core. Said pellets are further coated with atleast one layer comprising water insoluble release controlling agent and Paliperidone. These pellets are compressed into tablet with atleast one pharmaceutically acceptable excipients or filled in to capsule. Tablets are optionally coated with water insoluble release controlling coat.

It has also been found that paliperidone degrades into number of impurities. The major degradation products include C-9 ketone, N-oxides, and various dimers of its degradants. The stabilizing agents like butylated hydroxy toluene can be helpful in providing stability to the formulation.

In another embodiment, the instant invention is also designed to deliver the dosage form in extended release form in colonic region of the gastrointestinal (GI) tract in extended manner. The blood plasma concentration achieved with such formulation reduces the intraday tolerance effect developed.

The general procedure of manufacturing extended release solid oral pharmaceutical composition of Paliperidone is as follows:
a) Mixing of paliperidone and atleast one pharmaceutically acceptable excipient. The blend is then granulated, dried and then graded, so as to obtain the granules.
b) Mixing of the granules obtained in step a) with release controlling agent.
OR
a) Mixing of paliperidone, release controlling agent and atleast one pharmaceutically acceptable excipient. The blend is then granulated, dried and then graded, so as to obtain the granules.
b) Lubrication of the mixture obtained in step a) with lubricants.
c) Lubrication of the mixture obtained in step b) with lubricants.
d) Compression of the lubricated mixture obtained in step c) in to a tablet or filling in to capsule.
e) Optionally coating the compressed tablets from step d) with water insoluble release controlling agent.

The coating of the pellets or tablets can be carried out by any known method in the art, including spray application. Spraying can be carried out using fluidized bed processor or in a pan coating system. Alternatively coating can be done by hotmelt process using a granulator or fluidized bed processor or in a pan coating system or any other suitable process known to the skilled artisan.

Throughout this specification and the appended claims it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

### EXAMPLES

The present invention has been described by way of example only, and it is to be recognized that modifications thereto which fall within the scope and spirit of the appended claims, and which would be obvious to a skilled person based upon the disclosure herein, are also considered to be included within the invention.

### Example—1

TABLE I

| | | |
|---|---|---|
| S. No | Ingredients | 01 |

Core

| | | |
|---|---|---|
| 1. | Paliperidone (Micronized) | 9.00 |
| 2. | Pregelatinised Starch | 63.00 |
| 3. | Lactose Monohydrate | 60.28 |
| 4. | Butylated Hydroxy Toluene | 0.32 |
| 5. | Isopropyl Alcohol | q.s |
| 6. | Hypromellose (3 cps) | 5.25 |
| 7. | Poloxamer 188 | 2.10 |
| 8. | Purified Water | q.s |
| 9. | Stearic acid | 2.10 |
| 10. | Hypromellose (HPMC K4M CR) | 21.00 |
| 11. | Microcrystalline Cellulose | 46.95 |
| | Core Tablet Weight | 210.00 |

Coating

| | | |
|---|---|---|
| 1. | Eudragit ® FS 30 D * | 8.07 |
| 2. | Triethyl Citrate | 0.81 |
| 3. | Talc | 1.61 |
| 4. | Iron Oxide Red | 0.01 |
| 5. | Purified water | q.s |
| | Total Tablet Weight | 220.50 |

* Weight of dry polymer

Manufacturing Procedure:
1. Paliperidone and part quantity of Pregelatinised starch were mixed thoroughly.
2. Blend of step-1 was mixed with remaining quantity of Pregelatinised starch.
3. Blend of step-2 was mixed with Lactose monohydrate.
4. Butylated hydroxy toluene was dissolved in isopropyl alcohol and added over to blend from step-3.
5. Poloxamer 188 and Hypromellose (3cps) were dispersed in water. This binder preparation was used to granulate the blend from step-4. The wet granules were dried and then milled.
6. Hypromellose (HPMC K4M CR) and Microcrystalline cellulose were mixed with dried granules.
7. The blend from step-6 was mixed thoroughly with previously sifted stearic acid. The lubricated blend was compressed into tablets.
8. These tablets were coated with aqueous dispersion of Eudragit® FS 30 D, Triethyl citrate, Talc and Iron oxide red.

The dissolution profile of these tablets was found as below:

TABLE II

% Cumulative Paliperidone Released

| Time in | Invega ® 9 Tablet (B. No: | |
|---|---|---|
| hours | 6NA324; Mfgr: Alza) | 01 |
| 0 | 0.0 | 0.0 |
| 1 | 0.4 | 0.0 |
| 2 | 1.3 | 0.0 |
| 4 | 4.3 | 0.0 |
| 6 | 10.4 | 0.0 |
| 8 | 17.9 | 5.9 |
| 12 | 36.8 | 45.2 |
| 16 | 59.2 | 72.3 |
| 18 | 70.6 | 78.4 |
| 20 | 82.3 | 81.7 |
| 24 | 92.7 | 86.3 |

The dissolution details are as below:

| | |
|---|---|
| Apparatus | USP Type-I, Basket |
| RPM | 50 |
| Time Points (hrs) | |
| 0-1 hours | 750 ml, 0.1N HCl, |
| 1-6 hours | 900 mL, 6.5 pH Phosphate Buffer, |
| 6-24 hours | 1000 mL, 7.5 pH Phosphate Buffer, |

### Example—2

TABLE III

| | | |
|---|---|---|
| S. No | Ingredients | 02 |

Core

| | | |
|---|---|---|
| 1. | Paliperidone (Micronized) | 3.00 |
| 2. | Pregelatinised Starch | 66.00 |
| 3. | Lactose Monohydrate | 71.69 |
| 4. | Hypromellose (HPMC K4M CR) | 21.00 |
| 5. | Microcrystalline Cellulose | 38.55 |
| 6. | Hypromellose (3 cps) | 5.25 |
| 7. | Butylated Hydroxy Toluene | 0.32 |
| 8. | Isopropyl Alcohol | q.s |
| 9. | Poloxamer 188 | 2.10 |
| 10. | Purified Water | q.s |
| 11. | Stearic acid | 2.10 |
| | Core Tablet Weight | 210.00 |

Coating

| | | |
|---|---|---|
| 1. | Eudragit ® FS 30 D * | 8.07 |
| 2. | Triethyl Citrate | 0.81 |
| 3. | Talc | 1.62 |
| 4. | Purified water | q.s |
| | Total Tablet Weight | 220.50 |

* Weight of dry polymer

Manufacturing Procedure:
1. Paliperidone and part quantity of Pregelatinised starch were mixed thoroughly.
2. Blend of step-1 was mixed with remaining quantity of Pregelatinised starch.
3. Blend of step-2 was mixed with Lactose monohydrate.
4. Blend of step-3 was mixed with Hypromellose (HPMC K4M CR), Microcrystalline Cellulose and Hypromellose (3 cps)
5. Butylated hydroxy toluene was dissolved in isopropyl alcohol, dispersed Poloxamer 188, added purified water and mixed until to get clear solution. This binder preparation was used to granulate the blend from step-4. The wet granules were dried and then milled.
6. The blend from step-5 was mixed thoroughly with previously sifted stearic acid. The lubricated blend was compressed into tablets.
7. These tablets were coated with aqueous dispersion of Eudragit® FS 30 D, Triethyl citrate, Talc and Iron oxide red.

### Example 03 & 04

TABLE IV

| | | | |
|---|---|---|---|
| S. No | Ingredients | 03 | 04 |

Core

| | | | |
|---|---|---|---|
| 1. | Paliperidone | 9.00 | 9.00 |
| 2. | Hydroxyethyl Cellulose | 80.00 | 80.00 |
| 3. | Microcrystalline Cellulose | 162.97 | 162.97 |
| 4. | Butylated Hydroxy Toluene | 0.03 | 0.03 |
| 5. | Isopropyl Alcohol | q.s | q.s |
| 6. | Povidone | 9.00 | 9.00 |
| 7. | Purified Water | q.s | q.s |
| 8. | Magnesium Stearate | 5.00 | 5.00 |
| 9. | Colloidal Silicon Dioxide | 4.00 | 4.00 |
| | Core Tablet Weight | 270.00 | 270.00 |

Coating

| | | | |
|---|---|---|---|
| 1. | Kollicoat ® SR 30D * | — | 22.50 |
| 2. | Propylene Glycol | — | 2.25 |
| 3. | Talc | — | 4.88 |
| 4. | Iron Oxide Red | — | 0.37 |
| 5. | Purified water | — | q.s |
| | Total Tablet Weight | — | 300.00 |

* Weight of dry polymer

Manufacturing Procedure:
1. Paliperidone, Hydroxyethyl cellulose and Microcrystalline cellulose were mixed thoroughly.
2. Butylated hydroxy toluene was dissolved in isopropyl alcohol and added over blend from step-1.
3. Povidone was dissolved in water. This binder preparation was used to granulate the blend from step-2. The wet granules were dried and then milled.
4. The blend from step-3 was mixed thoroughly with previously sifted magnesium stearate and Colloidal silicon dioxide. The lubricated blend was compressed into tablets.
5. These tablets were coated (Example 4) with aqueous dispersion of Kollicoat® SR 30D, Propylene glycol, Talc and Iron oxide red.

### Example 05

TABLE V

| | | |
|---|---|---|
| S. No | Ingredients | 05 |

Core

| | | |
|---|---|---|
| 1. | Paliperidone (Micronized) | 3.00 |
| 2. | Pregelatinised Starch | 66.00 |
| 3. | Lactose Monohydrate | 71.69 |
| 4. | Hypromellose (HPMC K4M CR) | 21.00 |
| 5. | Microcrystalline Cellulose | 38.55 |
| 6. | Hypromellose (3 cps) | 5.25 |
| 7. | Butylated Hydroxy Toluene | 0.32 |
| 8. | Isopropyl Alcohol | q.s |
| 9. | Poloxamer 188 | 2.10 |
| 10. | Purified Water | q.s |
| 11. | Magnesium stearate | 2.10 |
| | Core Tablet Weight | 210.00 |

Coating

| | | |
|---|---|---|
| 1. | Eudragit ® FS 30 D * | 8.07 |
| 2. | Triethyl Citrate | 0.81 |
| 3. | Talc | 1.62 |
| 4. | Purified water | q.s |
| | Total Tablet Weight | 220.50 |

* Weight of dry polymer

Manufacturing Procedure:
1. Paliperidone and part quantity of Pregelatinised starch were mixed thoroughly.
2. Blend of step-1 was mixed with remaining quantity of Pregelatinised starch.
3. Blend of step-2 was mixed with Lactose monohydrate.
4. Blend of step-3 was mixed with Hypromellose (HPMC K4M CR), Microcrystalline Cellulose and Hypromellose (3 cps)
5. Butylated hydroxy toluene was dissolved in isopropyl alcohol, dispersed Poloxamer 188, added purified water and mixed until to get clear solution. This binder preparation was used to granulate the blend from step-4. The wet granules were dried and then milled.
6. The blend from step-5 was mixed thoroughly with previously sifted magnesium stearate. The lubricated blend was compressed into tablets.
7. These tablets were coated with aqueous dispersion of Eudragit® FS 30 D, Triethyl citrate, Talc and Iron oxide red.

1. An extended release solid oral pharmaceutical composition comprising Paliperidone or a pharmaceutically acceptable salt thereof, wherein the extended release composition comprises a matrix composition.

2. The solid oral pharmaceutical composition according to claim 1, wherein matrix agent is selected from a insoluble release controlling agent, a hydrophilic release controlling agent, a fatty release controlling agent or a combination thereof.

3. The solid oral pharmaceutical composition according to claim 1, wherein the said composition is coated with water insoluble release controlling agent.

4. The solid oral pharmaceutical composition according to claim 3, wherein the said coating layer comprises pH dependant water insoluble release controlling agent.

5. The solid oral pharmaceutical composition according to claim 3, wherein the said coating layer comprises pH independent water insoluble release controlling agent.

6. The solid oral pharmaceutical composition according to claim 2, wherein matrix agent is hydrophilic release controlling agent which is ionic or non-ionic.

7. The solid oral pharmaceutical composition according to claim 1, wherein the said composition further comprises wetting agent.

8. The solid oral pharmaceutical composition according to claim 6 wherein hydrophilic release controlling agent is selected from hydroxyethyl cellulose or hydroxyl propyl methyl cellulose.

9. The solid oral pharmaceutical composition according to claim 1 comprises a hydrophilic release controlling agent, wetting agent, stabilizing agent, wherein hydrophilic release controlling agent is added at intragranular stage.

10. The solid oral pharmaceutical composition according to claim 9, wherein the hydrophilic release controlling agent comprises hydroxypropyl methylcellulose, a xanthan gum or mixtures thereof.

11. The solid oral pharmaceutical composition according to claim 1, wherein the particle size of Paliperidone is 90% particles are less than 30 microns; preferably 90% particles are less than 20 microns, more preferably 90% particles are less than 10 microns.

12. An extended release solid oral pharmaceutical composition of Paliperidone or a pharmaceutically acceptable salt thereof, wherein the composition provides two different release profiles, wherein one release profile is delivered by an extended release matrix composition and another release profile comprises an immediate release or a second extended release in which Paliperidone is present and released from an outer coat.

13. An extended release solid oral pharmaceutical composition comprising pellets wherein the said pellets comprises paliperidone or its pharmaceutically acceptable salts and one or more release controlling agent optionally with pharmaceutically acceptable excipients.

14. The solid oral pharmaceutical composition according to claim 13, wherein the said pellets are compressed into tablet or filled in to capsule.

15. The solid oral pharmaceutical composition according to claim 14, wherein the tablet is coated with water insoluble release controlling agents.

16. The solid oral pharmaceutical composition according to claim 3, wherein the said coating layer of water insoluble release controlling agent further comprises Paliperidone or its pharmaceutically acceptable salt.

17. A process of preparing an extended release pharmaceutical composition of paliperidone comprises of following steps:
(a) mixing paliperidone, release controlling agent and at_least one pharmaceutically acceptable excipient. The blend is then granulated, dried and then graded, so as to obtain the granules.
(b) Lubrication of the mixture obtained in step a) with lubricants.
(c) Compression of the lubricated mixture obtained in step b) in a tablet or filling into capsule.
(d) Optionally coating the compressed tablets from step c) with water insoluble release controlling agent.

18. A process of preparing an extended release pharmaceutical composition of Paliperidone comprises of following steps:
a) Mixing of paliperidone or its pharmaceutically acceptable salt and at least one pharmaceutically acceptable excipient. The blend is then granulated, and then milled, so as to obtain the granules.
b) Mixing of the granules obtained in step a) with release controlling agent.
c) Lubrication of the mixture obtained in step b) with lubricants.
d) Compressing of the lubricated mixture obtained in step c) in a tablet or filling into capsule.
e) Optionally coating the compressed tablets from step d) with water insoluble release controlling agent
